(19) United States
(12) Patent Application Publication
(10) Pub. No.: US 2021/0401915 A1
(43) Pub. Date: Dec. 30, 2021

(54) COMPOSITIONS AND METHODS FOR THE MANAGEMENT OF MUCOSITIS IN MAMMALS

(71) Applicants: Andrew Kim, Newtown, PA (US); Carolyn Lammersfeld, Kenosha, WI (US)

(72) Inventors: Andrew Kim, Newtown, PA (US); Carolyn Lammersfeld, Kenosha, WI (US)

(21) Appl. No.: 17/474,036

(22) Filed: Sep. 13, 2021

Related U.S. Application Data
(60) Provisional application No. 63/078,198, filed on Sep. 14, 2020.

(51) Int. Cl. A61K 36/28; A61K 31/198; A61K 31/05; A61K 31/10; A61K 31/07; A61K 31/375; A61K 31/593; A61K 31/519; A61K 38/06; A61K 9/00; A61K 31/015; A61K 35/644; A61K 33/30

(52) U.S. Cl. CPC A61K 36/28; A61K 31/198; A61K 31/05; A61K 31/10; A61K 31/07; A61K 31/375; A61K 33/30; A61K 31/519; A61K 38/063; A61K 9/006; A61K 31/015; A61K 35/644; A61K 31/593

(57) ABSTRACT

Compositions comprising one or more nutritional substances are described, where the composition is suitable for oral consumption by a subject, and wherein the one or more compounds collectively provide the features of 1) interfering with the progression of mucositis at multiple points in the inflammatory cycle, while 2) promoting the structure, function, and integrity of the oral mucosa, and/or 3) inhibiting pathogenic bacterial, viral, or fungal overgrowth and/or 4) providing soothing relief to the patient. The compositions are useful for treating mucositis or xerostomia.

### TECHNICAL FIELD

The present disclosure relates to compositions and to methods of treatment using novel and non-obvious nutritional compositions consisting of specific proteins, dietary ingredients, plant extracts, food additives, vitamins, minerals, ingredients Generally Recognized as Safe by the United States Food and Drug Administration, and bee products for the management, which includes prevention and treatment of lesions to the oral mucosa, including ulcerative lesions and inflammatory lesions. More specifically, the present disclosure provides compositions and methods for the treatment of, for example, oral mucositis or xerostomia.

### BACKGROUND

Mucositis occurs in some subjects when cancer treatments and therapies break down rapidly dividing epithelial cells lining the gastro-intestinal tract (which is the tract from the mouth to the anus), leaving the mucosal tissue open to ulceration and infection. Mucosal tissue, also known as mucosa or the mucous membrane, lines all body passages that communicate with the air, such as the respiratory and alimentary tracts, and have cells and associated glands that secrete mucus. The part of this lining that covers the mouth, called the oral mucosa, is one of the most sensitive parts of the body and is particularly vulnerable to chemotherapy and radiation. The oral cavity is the most common location for mucositis.

Oral mucositis, also known as stomatitis, is a common, debilitating complication of cancer treatments, particularly chemotherapy and radiation. It is a frequent problem in bone marrow transplantation, contributing to increased morbidity and mortality. Oral mucositis can lead to several problems, including pain, nutritional deficiencies (as a result of inability to eat), and increased risk of infection due to open sores and ulcerations in the mucosa. It has a significant effect on the patient's quality of life and can be dose-limiting (e.g., requiring a reduction in subsequent chemotherapy doses).

Mucositis occurs in mammals, most notably in man, horses, and dogs. It consists of four distinct and well recognized stages, including a tissue inflammation and tissue damage phase, followed by an ulceration phase, a bacterial infection phase, a viral infection phase, and an epithelial proliferation phase. Accordingly, compositions, nutritional substances, and materials as disclosed herein may be used to ameliorate, reduce, and even eliminate one or more of the symptoms involving one or more of the phases of mucositis.

### BRIEF SUMMARY

In a first aspect, a composition is provided that comprises two or more nutritional substances that each individually display at least one of four desired effects, wherein the two or more nutritional substances act together to manage oral mucositis in mammals. The four desired effects comprise: (i) interference with an inflammatory cycle; (ii) promotion of the integrity of mucosal tissue; (iii) inhibition of the growth of a bacterium, a virus, a fungus, or any other pathogen or toxin derived thereof; and (iv) soothing agent. A “nutritional substance” is defined as a compound defined either as a dietary ingredient in US Public Law 103-417 (“Dietary Supplement Health and Education Act of 1994”); or as a food defined in section 201(f) of the Federal Food, Drug and Cosmetic Act (“FFDCA”), a food nutritional substance defined as Generally Recognized as Safe (“GRAS”) under sections 201(s) and 409 of the “FFDCA” as further defined by 21CFR 170.3, 21 CFR 170.30(b) and 170.30(c) and 170.3(f); or as an approved Food Ingredient, Food Additive, or Food Color as defined in subchapter 201(t) of the “FFDCA” and 21 CFR 70.3(f).

In a second aspect, a composition is provided that comprises two or more nutritional substances, wherein the two or more nutritional substances are selected from the nutritional substances listed on Table 1, the nutritional substances listed on Table 2, the nutritional substances listed on Table 3, and the nutritional substances listed on Table 4; and optionally, a carrier.

In an embodiment, the composition comprises three or more nutritional substances or four or more nutritional substances. In an embodiment, the composition is effective, when administered to a subject, to (j interfere with an inflammatory cycle; (ii) promote integrity of mucosal tissue; (iii) inhibit growth of a bacterium, a virus, a fungus, or any other pathogen or toxin derived thereof; and/or (iv) provide soothing relief from symptoms of mucositis or xerostomia.

In another aspect, a composition is provided that comprises a first nutritional substance with an activity profile comprised of one or more of the activities selected from the group consisting of (i) activity to modulate the inflammatory cycle, (ii) activity to promote integrity of mucosal tissue, (iii) activity to inhibit growth of a bacterium, a virus, a fungus or any other pathogen or toxin derived thereof, (iv) activity as a soothing agent; and a second nutritional substance with an activity profile comprised of one or more of the activities selected from the group consisting of (i) activity to interfere with an inflammatory cycle, (ii) to promote integrity of mucosal tissue, (iii) to inhibit growth of a bacterium or fungus, (iv) as a soothing agent. In an embodiment, the first nutritional substance activity profile may differ from the second nutritional substance activity profile, and together the activity profiles of the first nutritional substance and the second nutritional substance provide a composition with an activity profile that comprises the activities indicated as (i) and as (ii) and, optionally as (iii) or (iv).

In one embodiment, the composition further comprises a third nutritional substance with an activity profile comprised of one or more of the activities selected from the group consisting of (i) activity to interfere with an inflammatory cycle, (ii) to promote integrity of mucosal tissue, (iii) to inhibit growth of a bacterium, a virus, a fungus or any other pathogen or toxin derived thereof, (iv) as a soothing agent. The third nutritional substance activity profile may differ from the first nutritional substance and the second nutritional substance activity profiles, and together the activity profiles of the first nutritional substance, the second nutritional substance and the third nutritional substance provide a composition with an activity profile that comprises activities (i) and (ii) and (iii) or (iv).

In one embodiment, the composition further comprises a fourth nutritional substance with an activity profile comprised of one or more of the activities selected from the group consisting of (i) activity to interfere with an inflammatory cycle, (ii) to promote integrity of mucosal tissue, (iii) to inhibit growth of a bacterium, a virus, a fungus or any other pathogen or toxin derived thereof, (iv) as a soothing agent; wherein the fourth nutritional substance activity profile may differ from the activity profiles of the first nutritional substance, the second nutritional substance and the third nutritional substance. Together the activity profiles of the first nutritional substance, the second nutritional substance, the third nutritional substance and the fourth nutritional substance provide a composition with an activity profile that comprises activities (i) and (ii) and (iii) and (iv).

In one embodiment, the composition further comprises a carrier. In one embodiment, the carrier is a buffer or sterile water. In one embodiment, the carrier is an aqueous fluid.

In one embodiment, the composition is a solid. For example, the solid can be a frozen liquid, a powder, colloid or a lyophilate.

In one embodiment, the solid is reconstituted to form a liquid and then frozen.

In one embodiment, the composition is a liquid. For example, the liquid can be a gel or a suspension, solution or colloidal mixture.

In one embodiment, the liquid is frozen.

In one embodiment, the composition is safe for ingestion by a mammal.

In one embodiment, the composition contains no pharmaceutical drug compound, other than the nutritional substances identified herein, to the extent such are considered a pharmaceutical drug compound.

In one embodiment, the first nutritional substance is selected from the nutritional substances on Table A, or from the nutritional substances on Table B.

In one embodiment, at least one of the first nutritional substance, the second nutritional nutritional substance, and the fourth nutritional substance is selected from the nutritional substances listed on Table 5.

In one embodiment, at least one of the first nutritional substance, the second nutritional substance, and the third nutritional substance is selected from the nutritional substances listed in Table 6.

In one embodiment, at least one of the first nutritional substance, the third nutritional substance, and the fourth nutritional substance is selected from the nutritional substances listed in Table 7.

In one embodiment, at least one of the first nutritional substance and the second nutritional substance is selected from the nutritional substances listed in Table 8.

In one embodiment, at least one of the first nutritional substance and the third nutritional substance is selected from the nutritional substances listed in Table 9.

In one embodiment, at least one of the first nutritional substance and the fourth nutritional substance is selected from the nutritional substances listed in Table 10.

In another aspect, a method for treating oral mucositis is provided. The method comprises providing for administration, or administering, to an oral cavity of a subject a composition as described herein.

In another aspect, a method for mitigating oral mucositis or for slowing progression of oral mucositis is provided. The method comprises providing for administration, or administering, to an oral cavity of a subject a composition as described herein.

In another aspect, a method for ameliorating symptoms associated with oral mucositis in a subject is provided. The method comprises providing for administration, or administering, to an oral cavity of a subject a composition as described herein.

In one embodiment, the subject is in an initiation phase of oral mucositis with an initial injury to cells in the oral mucosa anticipated to be caused by radiotherapy and/or chemotherapy. In one embodiment, the subject is in a tissue inflammation and tissue damage phase of mucositis. In one embodiment, the subject is in an ulceration phase of mucositis. In one embodiment, the subject is in a bacterial infection phase of mucositis. In one embodiment, the subject is in an epithelial proliferation phase of mucositis.

In another aspect, a method for ameliorating symptoms associated with oral mucositis in a subject is provided. The method comprises providing for administration, or administering, to an oral cavity of a subject a composition as described herein, wherein the subject is in an initiation phase of oral mucositis.

In one embodiment, the composition provided for administration, or administered, to an oral cavity of a subject the composition is a reconstituted powder, a suspension, an oral gel, a topical spray, a solution, a colloidal liquid, packaged either in bulk or as a unit-of-use, palatable, freezable solution or suspension. The composition is administered, in one embodiment, via direct, topical application to the mucosa.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### BRIEF DESCRIPTION OF THE DRAWINGS

FIG. 1 illustrates an exemplary composition in the form of a solid dosage form.

FIG. 2 illustrates a method of using a composition, for the treatment of mucositis.

### DETAILED DESCRIPTION

### Definitions

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the revalues greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent.

The compositions of the present disclosure can comprise, consist essentially of, or consist of, the components disclosed.

All percentages, parts and ratios are based upon the total weight of the compositions and all measurements made are at about 25° C., unless otherwise specified.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The term “treating” is used herein, for instance, in reference to methods of treating a disorder, and generally includes the administration of a compound or composition which reduces the frequency of, or delays the onset of, symptoms of a medical condition in a subject relative to a subject not receiving the compound or composition. This can include reversing, reducing, or arresting the symptoms, clinical signs, and underlying pathology of a condition in a manner to improve or stabilize a subject's condition.

The terms “inhibiting” or “reducing” are used in reference to methods to inhibit or to reduce inflammation or lesions (e.g., decrease the size of or the number of oral lesions) in a population as compared to an untreated control population.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

A “nutritional substance” is defined as a compound defined either as a dietary ingredient in US Public Law 103-417 (“Dietary Supplement Health and Education Act of 1994”); or as a food defined in section 201(f) of the Federal Food, Drug and Cosmetic Act (“FFDCA”), a food nutritional substance defined as Generally Recognized as Safe (“GRAS”) under sections 201(s) and 409 of the “FFDCA” as further defined by 21CFR 170.3, 21 CFR 170.30(b) and 170.30(c) and 170.3(f); or as an approved Food Ingredient, Food Additive, or Food Color as defined in subchapter 201(t) of the “FFDCA” and 21 CFR 70.3(f).

### Compositions

Compositions comprising one or more compounds (also referred to as nutritional substances) are described, where the composition is suitable for oral consumption by a subject, and wherein the one or more compounds collectively provide the features of 1) interfering with the progression of mucositis at multiple points in the inflammatory cycle, while 2) promoting the structure, function, and integrity of the oral mucosa, and/or 3) inhibiting pathogenic bacterial, viral, or toxicogenic overgrowth and/or 4) providing soothing relief to the patient.

In some embodiments, a combination of nutritional substances is formulated for delivery in a powder for reconstitution, a suspension, an oral gel, a topical spray, or a liquid. In some embodiments, compositions as disclosed herein may be prepared in multiple dose packages, as a unit dosage form or unit-of-use, palatable, freezable solution or suspension (e.g., a “therapeutic ice pop”). The compositions provide for administration of complimentary compounds via direct, topical application to the mucosa, and for subsequent systemic absorption, for the treatment of mucositis. In some embodiments, the combination of compounds as disclosed herein may offer systemic absorption, which may be superior to other forms of delivery. In embodiments where the composition is a liquid that is frozen to be a solid, the composition provides cryotherapy in addition to the therapeutic benefit offered by the complimentary compounds.

In the following description of embodiments, different compositions will be described with reference to the compounds listed in Tables 1 through 11 and also including compounds listed in Tables A, B, C, and D, below.

In a first embodiment, a composition comprises a first nutritional substance selected from the nutritional substances listed on Table 1, a second nutritional substance selected from the nutritional substances listed on Table 2, a third nutritional substance selected from the nutritional substances listed on Table 3, a fourth nutritional substance selected from the nutritional substances listed on Table 4, and optionally, a carrier. Exemplary carriers are described infra.

In a second embodiment, a composition comprises a first nutritional substance with an activity profile having one or more of the activities selected from the group including (i) activity to modulate the inflammatory cycle, (ii) activity to promote integrity of mucosal tissue, (iii) activity to inhibit growth of a bacterium, a virus, a fungus or any other pathogen or toxin derived thereof, (iv) activity as a soothing agent, and a second nutritional substance with an activity profile including one or more of the activities selected from the group including (i) activity to interfere with an inflammatory cycle, (ii) to promote integrity of mucosal tissue, (iii) to inhibit growth of a bacterium or fungus, and (iv) as a soothing agent.

In some embodiments, the first nutritional substance activity profile differs from the second nutritional substance activity profile. In some embodiments, the activity profiles of the first nutritional substance and the second nutritional substance together may provide a composition with an activity profile that includes activities (i) and (ii) and, optionally (iii).

In some embodiments, the composition may include a third nutritional substance with an activity profile having one or more of the activities selected from the group including (i) activity to interfere with an inflammatory cycle, (ii) to promote integrity of mucosal tissue, (iii) to inhibit growth of a bacterium, a virus, a fungus or any other pathogen or toxin derived thereof, and (iv) as a soothing agent; wherein the third nutritional substance activity profile may differ from the first nutritional substance and the second nutritional substance activity profiles, and wherein together the activity profiles of the first nutritional substance, the second nutritional substance, and the third nutritional substance provide a composition with an activity profile that includes activities (i), (ii), and (iii).

In some embodiments, the composition may include a fourth nutritional substance with an activity profile including one or more of the activities selected from the group including (i) activity to interfere with an inflammatory cycle, (ii) to promote integrity of mucosal tissue, (iii) to inhibit growth of a bacterium, a virus, a fungus or any other pathogen or toxin derived thereof, and (iv) as a soothing agent. In some embodiments, the fourth nutritional substance activity profile differs from the activity profiles of the first nutritional substance, the second nutritional substance, and the third nutritional substance. In some embodiments, the activity profiles of the first nutritional substance, the second nutritional substance, the third nutritional substance, and the fourth nutritional substance provide, together, a composition with an activity profile that comprises activities (i), (ii), (iii), and (iv).

TABLE 1
Nutritional substances that interfere with inflammatory cycle

L-glutamine
20% water solution of cannabidiol (CBD)
Phytocannabinoids
Zinc L Carnosine
methylsulfonylmethane (MSM)
N-acetylcysteine (NAC)
Honey
Vitamin A
Vitamin C
Vitamin D
Folate
Beta Carotene
Glutathione (GSH)
Mixed Anthocyanadins
Grape Seed Extract
epigallocatechin-3-gallate (EGCG)

TABLE 2
Nutritional substances that promote structure/integrity of oral mucosa

L-glutamine
20% water solution of cannabidiol (CBD)
Phytocannabinoids
Zinc L Carnosine

TABLE 3
Nutritional substances that inhibit bacterial or fungal growth

Zinc L Carnosine
methylsulfonylmethane (MSM)
epigallocatechin-3-gallate (EGCG)

TABLE 4
Nutritonal Nutritional substances with activity as a soothing agent
(e.g., to soothe and/or relieve discomfort)

L-glutamine
20% water solution of cannabidiol (CBD)
Phytocannabinoids
methylsulfonylmethane (MSM)
Chamomile
Capsaicin
Honey

TABLE 5
Nutritional substances with a trifunctional activity profile of ability
to interfere with inflammatory cycle, promote structure/integrity of
oral mucosa, and soothe/relieve discomfort

L-glutamine
20% water solution of cannabidiol (CBD)
Phytocannabinoids

TABLE 6
Nutritional Nutritional substances with a trifunctional activity profile
of ability to interfere with inflammatory cycle, promote structure
and/or integrity of oral mucosa, and that inhibit a bacterial, viral
and/or fungal growth

Zinc L Carnosine
Vitamin D

TABLE 7
Nutritonal Nutritional substances with a trifunctional activity
profile of ability to interfere with inflammatory cycle, inhibit
bacterial, viral or fungal growth, and soothe/relieve discomfort
methylsulfonylmethane (MSM)

TABLE 8
Nutritonal Nutritional substances with a di-functional activity
profile of ability to interfere with an inflammatory cycle and
promote structure and/or integrity of an oral mucosa

Vitamin C
Folate
Beta Carotene
GSH

TABLE 9
Nutritional substances with a di-functional activity profile
of ability to interfere with inflammatory cycle and inhibit
bacterial, viral or fungal growth
EGCG

TABLE 10
Nutritonal Nutritional substances with a di-functional
activity profile of ability to promote structure/integrity of
oral mucosa and soothe/relieve discomfort
Capsaicin

TABLE 11
Nutritonal Nutritional substances with a di-functional
activity profile of ability to interfere with inflammatory cycle
and as a soothing agent (soothe/relieve discomfort)
Honey

TABLE A
GRAS Nutritional substances

L-glutamine
20% water solution of CBD
MSM
NAC
Honey, mint leaves, meadowsweet, clove, cinnamon,
blackcurreant leaves, bearberry,
Mixed Anthocyanadins
Grape Seed Extract
EGCG
Bee Propolis,
Curcumin
Calendula
Vitamin B12 (methylcobalamin)
Berry Extracts (flavoring)
Organic Stevia
Scutellaria Root-standardized
Chamomile
Capsaicin
Phytocannabinoids

TABLE B
Dietary Ingredient Nutritional substances

L-glutamine
NAC
Vitamin A
Vitamin C
Vitamin D
Folate from folic acid or 5-methyltetrandrofolate
Beta Carotene, grape seed extract, anthocyanadins,
GSH, nicotinamide, lemon balm, S-acetylglutthione
Melatonin
EGCG,
Whey protein
SAMe
P5P
Quecetin
Zinc L Carnosine
MSM
NAC
Curcumin
Berry Extracts
Calendula
Bee Propolis
Vitamin B12
Stevia
Honey

While Tables 1 through 11 or A and B provide compounds by specific names, it is understood that synonyms of each of the compounds listed in the tables may be known, and are intended. For example, some of the synonyms (but not limiting), may include the following:

L-Glutamine: (levo)glutamide; 2,5-Diamino-5-oxopentanoic acid; 2-Amino-4-carbamoylbutanoic acid; Endari.

Methyl sulfonylmethane: methyl sulfone; methyl sulfonylmethane; sulfonylbismethane; DMSO2.

N-acetylcysteine: N-acetylcysteine; N-acetyl-L-cysteine; NALC; NAC.

Folate: FA, N-(4-{[(2-amino-4-oxo-1,4-dihydropteridin-6-yl)methyl]amino}benzoyl)-L-glutamic acid, pteroyl-L-glutamic acid, folacin, vitamin B9, and historically, vitamin Bc and vitamin M.

Folic acid: dihydrofolate (DHF); tetrahydrofolate (THF); 5,10 methylenetetrahydrofolate (5,10-Methylene-THF); 5-methyltetrahydrofolate (5-MTHF).

Beta Carotene: Betacarotene (INN), β-Carotene, Food Orange 5, Provitamin A, 1,1′-(3,7,12,16-Tetramethyl-1,3,5,7,9,11,13,15,17-octadecanonaene-1,18-diyl)bis[2,6,6-trimethylcyclohexene].

Glutathione: γ-L-Glutamyl-L-cysteinylglycine; (2S)-2-Amino-5-[[(2R)-1-(carboxymethylamino)-1-oxo-3-sulfanylpropan-2-yl]amino]-5-oxopentanoic acid.

Epigallocatechin-3-gallate (EGCG): (−)-Epigallocatechin gallate; (2R,3R)-3′,4′,5,5′,7-pentahydroxyflavan-3-yl gallate.

Further, as can be seen from the listings in Tables 1 through 11, and A-B, many components may have multiple activities and play one or more roles in the disclosed composition. For example, the hemp-derived phytocannabinoids may act as a soothing compound based on their effect on the endocannabinoid system, which modulates both pain and inflammation. In some instances, a compound listed in one of the Tables may in fact perform other roles and have other synergistic effects in the disclosed composition, in addition to those properties and attributes listed in the table caption. For example, Chamomile and Capsaicin are listed in Table 4 as nutritional substances with activity as soothing agent. However, both of these two ingredients may also modulate the inflammatory phase in mucositis. In some embodiments, it is expected that Chamomile and Capsaicin may deliver relief via other mechanisms yet to be elucidated but already observed in historical use and various scientific reports.

In some embodiments, a composition as disclosed herein further includes a carrier. In some embodiments, the carrier is a buffer, sterile water, or an aqueous fluid.

In some embodiments, a composition as disclosed herein may be a solid. The solid may be a frozen liquid, a powder, or a lyophilizate. In some embodiments, the solid may be a frozen liquid from a reconstituted powder or lyophilizate. In some embodiments, the composition may be a liquid, such as a gel, colloidal mixture or a suspension. The liquid may be frozen. In some embodiments, the composition is safe for ingestion by a mammal (e.g., human, ape, monkey, a quadruped, and the like). In some embodiments, a composition as disclosed herein may be free, or mostly free of, pharmaceutical drug molecules.

In some embodiments, the first nutritional substance in the composition is selected from the nutritional substances on Table A. In some embodiments, the first nutritional substance in the composition is selected from the nutritional substances on Table B.

In some embodiments of the composition, at least one of the first nutritional substance, the second nutritional substance, and the fourth nutritional substance is selected from the nutritional substances listed on Table 5. In some embodiments, at least one of the first nutritional substance, the second nutritional substance, and the third nutritional substance is selected from the nutritional substances listed in Table 6. In some embodiments, at least one of the first nutritional substance, the third nutritional substance, and the fourth nutritional substance is selected from the nutritional substances listed in Table 7. In some embodiments, at least one of the first nutritional substance and the second nutritional substance is selected from the nutritional substances listed in Table 8. In some embodiments, at least one of the first nutritional substance and the third nutritional substance is selected from the nutritional substances listed in Table 9. In some embodiments, at least one of the first nutritional substance and the fourth nutritional substance is selected from the nutritional substances listed in Table 10.

In some embodiments, one of the first nutritional substances, the second nutritional substance, or the fourth nutritional substance in the composition is present in an amount that is sufficient to deliver to the subject an amount between about 500 mg to about 30 grams of L-glutamine. In some embodiments, a higher concentration of L-glutamine may be desirable, such as 10 g “ter-in-die” (three times a day, TID), or more. In embodiments when a finished product is dosed at 3-6 TID, a desirable amount of L-glutamine is within the range of 500 mg-1 gram

More specifically, some embodiments may include a higher end range with a single dose, so that when dosed 3-4 times daily, there is potential to get to 30 grams/day. When a finished product is dosed at 3-4 or even 6/day, a desirable amount of L-glutamine is within the range of 500 mg-30 grams. In some embodiments, a dosage of about to 2-30 grams/day in total daily dose may produce the desired therapeutic effect.

In some embodiments, one of the first nutritional substance, the second nutritional substance, or the fourth nutritional substance comprises up to about 30 grams of L-glutamine or any one of its synonyms, in a composition as disclosed herein. In some embodiments, one of the first nutritional substance, the second nutritional substance, or the fourth nutritional substance comprises between about 3 to 7 mg of 20% water soluble CBD, in a composition as disclosed herein. In some embodiments, one of the first nutritional substance, the third nutritional substance, or the fourth nutritional substance comprises between about 20 to 50 mg of MSM, in a composition as disclosed herein.

In some embodiments, one of the first nutritional substances may include between about 25-75 mg of NAC. In some embodiments, NAC is dosed at 1200 mg “bis-in-die” (twice a day, BID). In some embodiments, NAC increases glutathione (GSH). In some embodiments, GSH may have some oral bioavailability.

In some embodiments, a composition as disclosed herein may include s-acetyl-glutathione, which in concert with NAC and the anthocyanidins (cf. Table 1), may have a desirable therapeutic effect. In some embodiments, one of the first nutritional substance or the second nutritional substance may include between about 25-75 mg of glutathione (GSH), in a composition as disclosed herein. In some embodiments, the first nutritional substance comprises between about 10-50 mg of mixed anthocyanadins, in a composition as disclosed herein. In some embodiments, the first nutritional substance comprises between about 10-50 mg of mixed anthocyanadins, in a composition as disclosed herein. Food sources of anthocyanadins may include: blue, purple, and red colored plant foods, e.g., red and black grapes, cranberries, strawberries, blueberries, or red cabbage. Synonyms in compositions consistent with the present disclosure may include: proanthocyanidins, anthocyanidin, and anthocyanin.

In some embodiments, one of the fourth nutritional substance comprises between about 10-50 mg of chamomile, in a composition as disclosed herein. In some embodiments, one of the first nutritional substance comprises between about 50-100 μg of Vitamin A, in a composition as disclosed herein. In some embodiments, one of the first nutritional substance or the second nutritional substance comprises between about 1-50 mg of Vitamin C, in a composition as disclosed herein. In some embodiments, one of the first nutritional substance, the second nutritional substance, or the third nutritional substance comprises between about 1-20 μg of Vitamin D, in a composition as disclosed herein. In some embodiments, one of the first nutritional substance or the second nutritional substance comprises 15-60 μg of a folate selected from a folic acid or a methyl folate, in a composition as disclosed herein.

In some embodiments, a composition as disclosed herein may include Epigallocatechin gallate (EGCG, cf. Tables 3, 9, A and B), which has antibacterial properties, as does vitamin D (cf. Tables 1, 6, and B). In some embodiments, a composition as disclosed herein may include Cucumin, which is also antibacterial as are certain other botanicals.

In some embodiments, a composition as disclosed herein may include at least one of a nutritional substance selected from the group including of bee propolis; royal jelly; cucumina longa extracts; calendula officianalis extracts; vitamin B12 from methylcobalamin or cyanacobolamin; berry extracts from blueberries, strawberries, loganberries, cranberries, blackberries, cranberries, black currant (ribus nigrum), and edible juniper berries (e.g., juniperis californicus); high purity stevia glycoside extracts, including Rebiana (redaudioside A); S-adenosyl-L-methionine; whey protein; vitamin B6 from pyridoxine hydrochloride or pyridoxal 5′-Phosphate; quercitin; scutellaria baicalensis extracts and/or their isolates including baicalein, baicalin, wogonin, norwogonin, oroxylin A and beta-sitosterol; nicotinamide riboside; Melissa officianalis extract (Lemon Balm); Mentha balsamea Wild (peppermint); fillpendula ulmaria extract (meadowsweet); extracts of green mints (Lamiaceae) to include the culinary herbs, e.g., basil, rosemary, sage, marjoram, thyme, oregano; clove extracts; dwarf birch extracts, including Moxa (from Betula nana); cinnamon, preferably from C. venrum due to its lower coumarin content than C. cassia; bearberry fruit and leaf extract (from Artostaphylos alipina, Artostaphylos rubra, and/or Artostaphylos uva-ursi); Zingiber officinale root extract (ginger root); lycopene; resveratrol; valerian root extract; piper methysticum and/or piper wichmanii root extract (Kava); sodium bicarbonate; hyaluronic acid; grape seed extract; alpha lipoic acid.

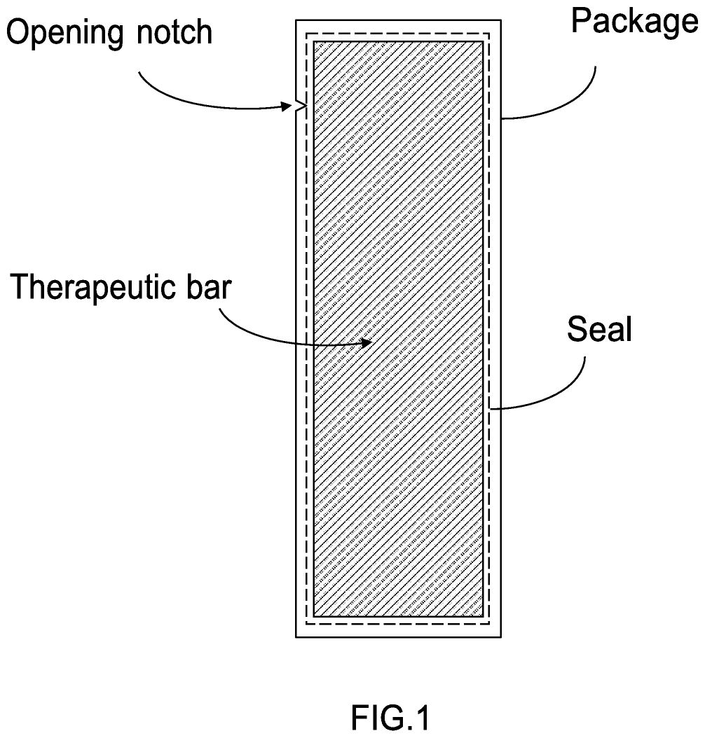

FIG. 1 illustrates an exemplary composition in the form of a therapeutic bar. The composition is enclosed in a package, which may include a seal to protect the therapeutic bar. The package may be a hermetic package configured to impede the passage of water, oxygen, and other compounds, including pathogens and larger contaminants to contact the therapeutic bar. In some embodiments, the package may include a cut, divot, or opening notch to facilitate the opening of the package and release of the therapeutic bar. The therapeutic bar may be in a solid form, such as a frozen liquid. In some embodiments, the therapeutic bar may be in a semi-solid form, such as a paste, a gel, a suspension, or a frozen paste. In some embodiments, the therapeutic bar may include components that make it palatable for a subject, such as nutritional additives, flavor additives, and even colorant additives.

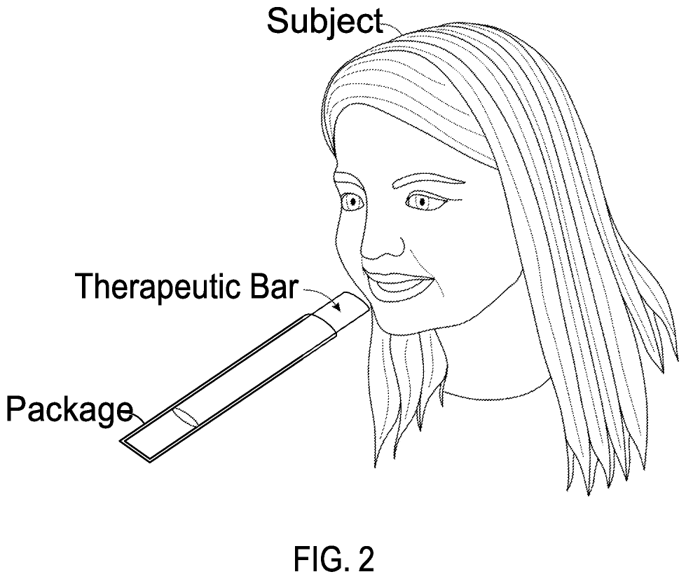

FIG. 2 illustrates a method of providing a compound as disclosed herein to a subject, for the treatment of mucositis, according to some embodiments. Accordingly, the subject opens the package on one and pulls at least a portion of the therapeutic bar out, to put it in her mouth, for consumption.

### II. Methods of Treatment

Also contemplated are methods for using the compositions for treating a subject. Methods for treating mucositis, and in particular oral mucositis, are provided, where a composition is provided for administration, or is administered, to an oral cavity of a subject.

In some embodiments, a method for mitigating oral mucositis or for slowing progression of oral mucositis, includes providing for administration or administering to an oral cavity of a subject a composition as disclosed herein.

In some embodiments, a method for ameliorating symptoms associated with oral mucositis in a subject includes providing for administration, or administering, to an oral cavity of a subject a composition as disclosed herein.

In some embodiments, a method consistent with the present disclosure is performed when the subject is in an initiation phase of oral mucositis at high risk for an initial injury to cells in the oral mucosa caused by radiotherapy and/or chemotherapy.

In some embodiments, a method consistent with the present disclosure is performed when the subject is in a tissue inflammation and tissue damage phase of mucositis. In some embodiments, a method consistent with the present disclosure is performed when the subject is in an ulceration phase of mucositis. In some embodiments, a method consistent with the present disclosure is performed when the subject is in a bacterial infection phase of mucositis. In some embodiments, a method consistent with the present disclosure is performed when the subject is in an epithelial proliferation phase of mucositis.

In some embodiments, a method for ameliorating symptoms associated with oral mucositis in a subject includes providing for administration or administering to an oral cavity of a subject a composition as disclosed herein, when the subject is in an initiation phase of oral mucositis.

Also contemplated are methods for treating xerostomia. The methods comprise providing for administration or administering to an oral cavity of a subject a composition as described herein. Xerostomia, or dry mouth, can range from mild oral discomfort to a severe condition that compromises health, dietary intake and quality of life. The method of treatment contemplated herein, in one embodiment, is for treating dry mouth associated with chemotherapy or radiotherapy, particularly head and/or neck radiotherapy. In other embodiments, the method of treatment of dry mouth is for treating dry mouth associated with an autoimmune disease or other condition, such as hormonal changes or diabetes. The method is effective to improve salivary flow and relieving discomfort associated with the dry mouth.

In the methods for treating oral mucositis and/or xerostomia, provided for administration or administered to an oral cavity of a subject is composition as disclosed herein which, in some embodiments, can be at least one of a reconstituted powder, a suspension, an oral gel, a topical spray, a liquid, or as a unit-of-use, palatable, freezable solution or suspension comprising the composition via direct, topical application.

### EXAMPLES

The following examples are illustrative in nature and are in no way intended to be limiting.

### Exemplary Composition

An exemplary composition consistent with the present disclosure includes: 5-10 grams L-glutamine, equivalent of 1.5 teaspoon to IT honey, 5-10 mgs zinc, capsaicin with addition of vitamins D, A, C, folate, beta carotene.

### Exemplary Composition

An exemplary composition consistent with the present disclosure includes: 5-10 grams L-glutamine, equivalent of 1.5 teaspoon to IT honey, 5-10 mgs zinc, chamomile with addition of vitamins D, A, C, folate, beta carotene.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public, regardless of whether such disclosure is explicitly recited in the above description. No clause element is to be construed under the provisions of 35 U.S.C. § 112, sixth paragraph, unless the element is expressly recited using the phrase “means for” or, in the case of a method clause, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following clauses. For example, while operations are depicted in the drawings in a particular order, this should not be understood as requiring that such operations be performed in the particular order shown or in sequential order, or that all illustrated operations be performed, to achieve desirable results. The actions recited in the clauses can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the clauses. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein, but are to be accorded the full scope consistent with the language claims and to encompass all legal equivalents. Notwithstanding, none of the clauses are intended to embrace subject matter that fails to satisfy the requirements of the applicable patent law, nor should they be interpreted in such a way.

What is claimed is:

1. A novel and non-obvious nutritional composition, comprising:
two or more nutritional substances that each individually display at least one of four desired effects, wherein the two or more nutritional substances act together to manage oral mucositis in a mammal, and wherein the four desired effects comprise:
(i) interference with an inflammatory cycle;
(ii) promotion of the integrity of mucosal tissue;
(iii) inhibition of the growth of a bacterium, a virus or a fungus; and
(iv) soothing agent.

2. A novel and non-obvious nutritional composition, comprising: two or more nutritional substances, wherein the two or more nutritional substances are selected from the nutritional substances listed on Table 1, the nutritional substances listed on Table 2, the nutritional substances listed on Table 3, and the nutritional substances listed on Table 4.

3. The composition of claim 2, wherein the composition is effective, when administered to a subject, to (i) interfere with an inflammatory cycle; (ii) promote integrity of mucosal tissue; (iii) inhibit growth of a bacterium, a virus or a fungus; and/or (iv) soothe a lesion.

4. A composition, comprising:
a first nutritional substance selected from the nutritional substances listed on Table 1, Table 2, Table 3 and Table 4;
a second nutritional substance selected from the nutritional substances listed on Table 1, Table 2, Table 3 and Table 4;
optionally, a third nutritional substance selected from the nutritional substances listed on Table 1, Table 2, Table 3 and Table 4; and
optionally, a fourth nutritional substance selected from the nutritional substances listed on Table 1, Table 2, Table 3 and Table 4.

5. A composition, comprising:
a first nutritional substance with an activity profile comprised of one or more of the activities selected from the group consisting of (i) activity to modulate the inflammatory cycle, (ii) activity to promote integrity of mucosal tissue, (iii) activity to inhibit growth of a bacterium, a virus, or a fungus, (iv) activity as a soothing agent; and
a second nutritional substance with an activity profile comprised of one or more of the activities selected from the group consisting of (i) activity to interfere with an inflammatory cycle, (ii) to promote integrity of mucosal tissue, (iii) to inhibit growth of a bacterium, a virus or a fungus, (iv) as a soothing agent;
wherein the first nutritional substance activity profile differs from the second nutritional substance activity profile, and wherein together the activity profiles of the first nutritional substance and the second nutritional substance provide a composition with an activity profile that comprises activities (i) and (ii) and, optionally (iii).

6. The composition of claim 6, further comprising a third nutritional substance with an activity profile comprised of one or more of the activities selected from the group consisting of (i) activity to interfere with an inflammatory cycle, (ii) to promote integrity of mucosal tissue, (iii) to inhibit growth of a bacterium, a virus or a fungus, (iv) as a soothing agent; wherein the third nutritional substance activity profile differs from the first nutritional substance and the second nutritional substance activity profiles, and wherein together the activity profiles of the first nutritional substance, the second nutritional substance and the third nutritional substance provide a composition with an activity profile that comprises activities (i) and (ii) and (iii).

7. The composition of claim 6, further comprising a fourth nutritional substance with an activity profile comprised of one or more of the activities selected from the group consisting of (i) activity to interfere with an inflammatory cycle, (ii) to promote integrity of mucosal tissue, (iii) to inhibit growth of a bacterium, a virus or a fungus, (iv) as a soothing agent; wherein the fourth nutritional substance activity profile differs from the activity profiles of the first nutritional substance, the second nutritional substance and the third nutritional substance, and wherein together the activity profiles of the first nutritional substance, the second nutritional substance, the third nutritional substance and the fourth nutritional substance provide a composition with an activity profile that comprises activities (i) and (ii) and (iii) and (iv).

8. The composition of any one of claims 1-7, further comprising a carrier.

9. The composition of claim 8, wherein the carrier is a buffer or sterile water.

10. The composition of claim 8, wherein the carrier is an aqueous fluid.

11. The composition of any one of claims 1-8, wherein the composition is a solid.

12. The composition of claim 11, wherein the solid is a frozen liquid.

13. The composition of claim 11, wherein the solid is a powder or lyophilizate.

14. The composition of claim 13, wherein the solid is reconstituted to form a liquid and then frozen.

15. The composition of any one of claims 1-10, wherein the composition is a liquid.

16. The composition of claim 15, wherein the liquid is a gel or a suspension.

17. The composition of claim 15 or 16, wherein the liquid is frozen.

18. The composition of any preceding claim, wherein the composition is safe for ingestion by a mammal.

19. The composition of any preceding claim, containing no pharmaceutical drug molecules.

20. The composition of any preceding claim, wherein the first nutritional substance is selected from the nutritional substances on Table A.

21. The composition of any preceding claim where in the first nutritional substance is selected from the nutritional substances on Table B.

22. The composition of any one of claims 1 and 9-18, wherein at least one of the first nutritional substance, the second nutritional substance, and the fourth nutritional substance is selected from the nutritional substances listed on Table 5.

23. The composition of any one of claims 1 and 9-18, wherein at least one of the first nutritional substance, the second nutritional substance, and the third nutritional substance is selected from the nutritional substances listed in Table 6.

24. The composition of any one of claims 1 and 9-18, wherein at least one of the first nutritional substance, the third nutritional substance, and the fourth nutritional substance is selected from the nutritional substances listed in Table 7.

25. The composition of any one of claims 1 and 9-18, wherein at least one of the first nutritional substance and the second nutritional substance is selected from the nutritional substances listed in Table 8.

26. The composition of any one of claims 1 and 9-18, wherein at least one of the first nutritional substance and the third nutritional substance is selected from the nutritional substances listed in Table 9.

27. The composition of any one of claims 1 and 9-18, wherein at least one of the first nutritional substance and the fourth nutritional substance is selected from the nutritional substances listed in Table 10.

28. The composition of any one of claims 1 and 9-18, wherein one of the first nutritional substance, the second nutritional substance, or the fourth nutritional substance comprises between about 500 mg to about 30 grams of L-glutamine.

29. The composition of any one of claims 1 and 9-18, wherein one of the first nutritional substance, the second nutritional substance, or the fourth nutritional substance comprises up to about 30 grams of L-glutamine or any one of its synonyms.

30. The composition of any one of claims 1 and 9-18, wherein one of the first nutritional substance, the second nutritional substance, or the fourth nutritional substance comprises between about 3 to 7 mg of 20% water soluble CBD.

31. The composition of any one of claims 1 and 9-18, wherein one of the first nutritional substance, the third nutritional substance, or the fourth nutritional substance comprises between about 20 to 50 mg of MSM.

32. The composition of any one of claims 1 and 9-18, wherein one of the first nutritional substance comprises between about 25-75 mg of NAC.

33. The composition of any one of claims 1 and 9-18, wherein one of the fourth nutritional substance comprises between about 10-50 mg of chamomile.

34. The composition of any one of claims 1 and 9-18, wherein one of the first nutritional substance comprises between about 50-100 μg of Vitamin A.

35. The composition of any one of claims 1 and 9-18, wherein one of the first nutritional substance or the second nutritional substance comprises between about 1-50 mg of Vitamin C.

36. The composition of any one of claims 1 and 9-18, wherein one of the first nutritional substance, the second nutritional substance or the third nutritional substance comprises between about 1-20 μg of Vitamin D.

37. The composition of any one of claims 1 and 9-18, wherein one of the first nutritional substance or the second nutritional substance comprises 15-60 μg of a folate selected from a folic acid or a methyl folate.

38. The composition of any one of claims 1 and 9-18, wherein one of the first nutritional substance or the second nutritional substance comprises between about 25-75 mg of glutathione (GSH).

39. The composition of any one of claims 1 and 9-18, wherein the first nutritional substance comprises between about 10-50 mg of mixed anthocyanadins.

40. The composition of any one of claims 1 and 9-18, further comprising at least one nutritional substance selected from the group consisting of bee propolis; royal jelly; cucumina longa extracts; calendula officianalis extract; vitamin B12 from methylcobalamin, vitamin B12 from cyanacobolamin; a berry extract from: blueberries, strawberries, loganberries, cranberries, blackberries, black currant (ribus nigrum), edible juniper berries (juniperis californicus); stevia glycoside extracts including Rebiana or redaudioside A; S-adenosyl-L-methionine; whey protein; vitamin B6 from pyridoxine hydrochloride or from pyridoxal 5′-Phosphate; quercitin; scutellaria baicalensis extracts and their isolates including: baicalein, baicalin, wogonin, norwogonin, oroxylin A and beta-sitosterol; nicotinamide riboside; Melissa officianalis extract or Lemon Balm; Mentha balsamea Wild or peppermint; fillpendula ulmaria extract or meadowsweet; extracts of green mints or Lamiaceae including culinary herbs comprising one of: basil, rosemary, sage, marjoram, thyme, and oregano; clove extracts; dwarf birch extracts, including Moxa from Betula nana; cinnamon, preferably from C. venrum; bearberry fruit and leaf extract from: Artostaphylos alipina, Artostaphylos rubra, or Artostaphylos uva-ursi; Zingiber officinale root extract or ginger root; lycopene; resveratrol; valerian root extract; piper methysticum; piper wichmanii root extract or Kava; sodium bicarbonate; hyaluronic acid; grape seed extract; and alpha lipoic acid.

41. A method for treating oral mucositis, comprising:
providing for administration or administering to an oral cavity of a subject a composition of any one of claims 1-40.

42. A method for mitigating oral mucositis or for slowing progression of oral mucositis, comprising:
providing for administration or administering to an oral cavity of a subject a composition of any one of claims 1-40.

43. A method for ameliorating symptoms associated with oral mucositis in a subject, comprising:
providing for administration or administering to an oral cavity of a subject a composition of any one of claims 1-40.

44. The method of claim 43, wherein the subject is in an initiation phase of oral mucositis with an initial injury to cells in the oral mucosa caused by radiotherapy and/or chemotherapy.

45. The method of claim 43, wherein the subject is in a tissue inflammation and tissue damage phase of mucositis.

46. The method of claim 43, wherein the subject is in an ulceration phase of mucositis.

47. The method of claim 43, wherein the subject is in a bacterial infection phase of mucositis.

48. The method of claim 43, wherein the subject is in an epithelial proliferation phase of mucositis.

49. A method for ameliorating symptoms associated with oral mucositis in a subject, comprising:
providing for administration or administering to an oral cavity of a subject a composition of any one of claims 1-40 wherein the subject is in an initiation phase of oral mucositis.

50. The method of any one of claims 43-49, wherein providing for administration or administering to an oral cavity of a subject the composition comprises providing at least one of a reconstituted powder, a suspension, an oral gel, a topical spray, a liquid, or as a unit-of-use, palatable, freezable solution or suspension comprising the composition via direct, topical application.

51. A method for treating xerostomia, comprising providing for administration or administering to an oral cavity of a subject the composition of any one of claims 1-40.
